(19) United States
(12) Patent Application Publication
(10) Pub. No.: US 2019/0388359 A1
(43) Pub. Date: Dec. 26, 2019

(54) METHODS OF PROMOTING THE HEALING OF CONCAVE WOUNDS

(71) Applicant: NANOFIBER SOLUTIONS, LLC, Hilliard, OH (US)

(72) Inventors: Jed JOHNSON, London, OH (US); Jason CHAKROFF, Columbus, OH (US)

(21) Appl. No.: 16/453,230

(22) Filed: Jun. 26, 2019

Related U.S. Application Data
(60) Provisional application No. 62/689,998, filed on Jun. 26, 2018.

(51) Int. Cl. A61K 9/70; D01F 1/10; D01D 5/00

(52) U.S. Cl. CPC A61K 9/70; A61L 15/40; D01D 5/0076; D01F 1/10

(57) ABSTRACT

The instant disclosure is directed to methods of promoting the healing of concave wounds. A method for promoting the healing of a concave wound may comprise placing a scaffold over the concave wound, the scaffold comprising an electrospun polymer fiber. The scaffold may be pressed into the concave wound, such that the scaffold simultaneously contacts at least a bottom portion of the concave wound and at least a side portion of the concave wound. The method may be suture free. The scaffold employed in such a method may have an asterisk shape comprising a center and at least two spokes, and pressing the scaffold into the concave wound may comprise pressing the center of the scaffold into the bottom portion of the concave wound, and allowing the at least two spokes of the scaffold to fold upward to contact the side portion of the concave wound.

### CROSS-REFERENCE TO RELATED APPLICATIONS

This application claims priority to and benefit of U.S. Provisional Application Ser. No. 62/689,998, filed Jun. 26, 2018, entitled “Methods of Promoting the Healing of Concave Wounds,” which is incorporated herein by reference in its entirety.

### BACKGROUND

Concave wounds, produced both as the result of medical procedures, diseases, and the result of traumatic injuries, are common. Such wounds may be difficult to heal using conventional treatments. For example, when a punch biopsy is taken from a patient's skin or muscle tissue, the site of the punch biopsy does not heal readily. Conventional treatments for a punch biopsy site include cleansing the wound, applying an antibiotic ointment, and in some cases, suturing the edges of the site together. When such treatments are used, however, a patient may be required to attend follow-up appointments in which the sutures are removed and other treatments may be applied. In the case of chronic wounds, treatment includes debridement in addition to normal cleaning and wound management. These types of wounds can become cavernous and progress through the full thickness of the skin to the bone. None of these conventional treatments represents an optimal approach to managing these types of wounds. Thus, there exists a need for a method of promoting the healing of concave wounds that is simple, effective, maintains uniform contact with the wound bed, and does not necessarily require follow-up physician visits.

### SUMMARY

The instant disclosure is directed to methods of promoting the healing of concave wounds. In one embodiment, a method for promoting the healing of a concave wound may comprise placing a scaffold over the concave wound. In certain embodiments, the scaffold may comprise an electrospun polymer fiber. In an embodiment, the method may further include pressing the scaffold into the concave wound, such that the scaffold simultaneously contacts at least a bottom portion of the concave wound and at least a side portion of the concave wound. In some embodiments, the method may be suture free. In certain embodiments, the scaffold may have an asterisk shape comprising a center and at least two spokes, and pressing the scaffold into the concave wound may comprise pressing the center of the scaffold into the bottom portion of the concave wound, and allowing the at least two spokes of the scaffold to fold upward to contact the side portion of the concave wound.

In one embodiment, a scaffold for promoting the healing of a concave wound may comprise an electrospun polymer fiber. In certain embodiments, the scaffold may have an asterisk shape comprising a center and at least two spokes; in other embodiments, the scaffold may have a “cupcake liner” shape comprising a flat center and at least one wall substantially perpendicular to the center. Further embodiments of the instant disclosure are described herein.

### BRIEF DESCRIPTION OF THE DRAWINGS

FIG. 1A illustrates the step of placing a scaffold over a model of a concave wound, in accordance with the present disclosure.

FIG. 1B illustrates the step of pressing the scaffold of FIG. 1A into the model of the concave wound, in accordance with the present disclosure.

FIG. 1C illustrates the scaffold of FIG. 1A and FIG. 1B simultaneously contacting at least a bottom portion of the model of the concave wound and at least a side portion of the model of the concave wound, in accordance with the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

The terms “animal,” “patient,” “mammal,” and “subject” as used herein include, but are not limited to, humans and non-human vertebrates such as wild, domestic, and farm animals. In some embodiments, the terms “animal,” “patient,” “mammal,” and “subject” may refer to humans.

As used herein, the term “biocompatible” refers to non-harmful compatibility with living tissue. Biocompatibility is a broad term that describes a number of materials, including bioinert materials, bioactive materials, bioabsorbable materials, biostable materials, biotolerant materials, or any combination thereof.

As used herein, the term “concave wound” refers to any internal or external wound on or in any portion of any patient or subject, in which at least a portion of the wound is sunken or depressed as compared to a comparable non-wounded portion of that patient or subject. A concave wound may include, for example, a puncture wound, a cavernous wound, a traumatic wound, a blast wound, a chronic wound, an ulcer (including, for example, a diabetic ulcer, a venous stasis ulcer, an arterial insufficiency ulcer, and/or a pressure ulcer), a caustic wound, a thermal wound, a surgical wound, a biopsy site, a punch biopsy site, an acute wound, a chronic wound, a partial-thickness wound, a full-thickness wound, a tunneled or undermined wound, combinations thereof, and the like.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including the repair or replacement of biological structures. In some embodiments, the resulting fiber scaffold may be implanted into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

In certain embodiments, a first polymer solution comprising a first polymer and a second polymer solution comprising a second polymer may each be used in a separate polymer injection system at substantially the same time to produce one or more electrospun fibers comprising the first polymer interspersed with one or more electrospun fibers comprising the second polymer. Such a process may be referred to as “co-spinning” or “co-electrospinning,” and a scaffold produced by such a process may be described as a co-spun or co-electrospun scaffold.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 5 μm, about 10 μm, about 20 μm, or ranges between any two of these values, including endpoints. In some embodiments, the electrospun fiber diameter may be from about 0.25 μm to about 20 μm.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, polyethylene oxide terephthalate, polybutylene terephthalate, polyethylene oxide terephthalate-co-polybutylene terephthalate, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints. In one embodiment, the polymer solution may include tantalum present in an amount of about 10 wt % to about 1,500 wt %.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time. Such fibers may act as an initial template or scaffold during a healing process. These templates or scaffolds may degrade in vivo once the tissues have a degree of healing by natural structures and cells. It may be further understood that a polymer solution and its resulting electrospun fiber(s) may be composed or more than one type of polymer, and that each polymer therein may have a specific characteristic, such as bio-stability, biodegradability, or bioabsorbability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Scaffolds for Promoting the Healing of Concave Wounds

The instant disclosure is directed to methods of promoting the healing of concave wounds. It may be understood that the devices and methods described herein may be applied to any concave wound of any internal or external tissue of any subject or patient, and that the examples described herein are non-limiting.

Concave wounds, produced both as the result of medical procedures, diseases, and the result of traumatic injuries, are common. Such wounds may be difficult to heal using conventional treatments. For example, when a punch biopsy is taken from a patient's skin or muscle tissue, the site of the punch biopsy does not heal readily. Conventional treatments for a punch biopsy site include cleansing the wound, applying an antibiotic ointment, and in some cases, suturing the edges of the site together. When such treatments are used, however, a patient may be required to attend follow-up appointments in which the sutures are removed and other treatments may be applied. In the case of chronic wounds, treatment includes debridement in addition to normal cleaning and wound management. These types of wounds can become cavernous and progress through the full thickness of the skin to the bone. None of these conventional treatments represents an optimal approach to managing these types of wounds. Thus, there exists a need for a method of promoting the healing of concave wounds that is simple, effective, maintains uniform contact with the wound bed, and does not necessarily require follow-up physician visits.

A device for promoting the healing of a concave wound may comprise a scaffold, and the scaffold may include one or more electrospun polymer fibers. In certain embodiments, the scaffold may have a shape that allows it to conform to the morphology of a concave wound or a portion thereof.

In certain embodiments, the one or more electrospun polymer fibers of the scaffold may comprise one or more polymers. The polymers may comprise, for example, polyethylene oxide terephthalate, polybutylene terephthalate, polyethylene oxide terephthalate-co-polybutylene terephthalate, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof. In some embodiments, the one or more electrospun polymer fibers of the scaffold may comprise one or more fibers comprising different polymers that have been co-electrospun. In one embodiment, the one or more electrospun polymer fibers of the scaffold may comprise co-electrospun polyglycolic acid and polylactide-co-caprolactone.

In some embodiments, for example, the scaffold may have a shape of an asterisk. In certain embodiments, the asterisk may have a center and at least two “spokes.” The asterisk may have, for example, three spokes, four spokes, five spokes, six spokes, seven spokes, eight spokes, nine spokes, ten spokes, eleven spokes, twelve spokes, or any range between any two of these values, including endpoints. In embodiments wherein the scaffold has the shape of an asterisk, the center of the asterisk may have a diameter of about 1 mm to about 300 mm. The center of the asterisk may have a diameter of, for example, about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, or any range between any two of these values, including endpoints. In certain embodiments, the diameter of the center of the asterisk may correspond or substantially correspond with a diameter or other dimension of the concave wound it is being used to treat. In some embodiments, the diameter of the center of the asterisk may be customized, manually or during manufacturing, to correspond or substantially correspond with a diameter or other dimension of the concave wound it is being used to treat.

In embodiments wherein the scaffold has the shape of an asterisk, each of the spokes may independently have a length of about 1 mm to about 300 mm. The length of each spoke may independently be, for example, about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, or any range between any two of these values, including endpoints. In certain embodiments, the length of the spokes of the asterisk may correspond or substantially correspond with a depth or other measurement of the concave wound it is being used to treat. In some embodiments, the spokes of the asterisk may be customized, manually or during manufacturing, to correspond or substantially correspond with a depth or other dimension of the concave wound it is being used to treat.

In other embodiments, the scaffold may have the shape of a “cupcake liner.” In some embodiments, a scaffold having the shape of a cupcake liner may have a shape such resembling a concave depression, a blister, or the like. In certain embodiments, the scaffold having the shape of a cupcake liner may comprise a flat center and at least one wall. In one embodiment, the wall may be situated substantially perpendicular to the center; in another embodiment, the wall may be situated at an angle of at least about 30 degrees from the center. In still other embodiments, the wall may be situated at an angle of at least about 45 degrees from the center. In certain embodiments, the wall may have an accordion-like structure, while in other embodiments, the wall may have a structure that is substantially smooth at the macro level.

In embodiments wherein the scaffold has the shape of a cupcake liner, the center of the cupcake liner may have a diameter of about 1 mm to about 300 mm. The center of the cupcake liner may have a diameter of, for example, about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, or any range between any two of these values, including endpoints. In one embodiment, the center of the cupcake liner may have a diameter of about 4 mm. In certain embodiments, the diameter of the center of the cupcake liner may correspond or substantially correspond with a diameter or other measurement of the concave wound it is being used to treat. In some embodiments, the diameter of the center of the cupcake liner may be customized, manually or during manufacturing, to correspond or substantially correspond with a diameter or other dimension of the concave wound it is being used to treat.

In embodiments wherein the scaffold has the shape of an cupcake liner, the wall of the cupcake liner may have a height of about 1 mm to about 300 mm. The height of the wall may be, for example, about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, or any range between any two of these values, including endpoints. In one embodiment, the wall of the cupcake liner may have a height of about 4 mm. In certain embodiments, the height of the wall of the cupcake liner may correspond or substantially correspond with a depth or other measurement of the concave wound it is being used to treat. In some embodiments, the height of the wall of the cupcake liner may be customized, manually or during manufacturing, to correspond or substantially correspond with a depth or other dimension of the concave wound it is being used to treat.

In certain embodiments, the shape of the scaffold, or one or more dimensions of the scaffold may be customized to correspond or substantially correspond with a dimension of the concave wound it is being used to treat. The dimensions of the concave wound may include, for example, a diameter, a depth, a width, a length, or a custom morphology of a complex wound. In some embodiments, the depth of the concave wound may be at least 1 mm. In other embodiments, the depth of the concave wound may be at least 2 mm.

In some embodiments, the dimensions of the concave wound may also be expressed in a width-to-depth ratio. In one non-limiting example, if a wound is about 1 mm wide and about 4 mm deep, the wound's width-to-depth ratio would be about 1:4. The width-to-depth ratio for a particular concave wound may range from about 10:1 to about 1:10. The width-to-depth ratio may be, for example, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, or any range between any two of these values, including endpoints.

In some embodiments, the scaffold may have any shape that allows it to be configured to simultaneously or substantially simultaneously contact at least a bottom portion of a concave wound and at least one side portion of the concave wound. In certain embodiments, the scaffold may be configured to simultaneously or substantially contact between about 50% and about 100% of a bottom portion of the concave wound and between about 50% and about 100% of a side portion of the concave wound. In some embodiments, the scaffold may be configured to contact, for example, about 50%, about 60%, about 70%, about 80%, about 90%, about 100%, or any range between any two of these percentages, of a bottom portion of a concave wound, while simultaneously or substantially simultaneously contacting, for example, about 50%, about 60%, about 70%, about 80%, about 90%, about 100%, or any range between any two of these percentages, of a side portion of the concave wound.

### Methods for Promoting the Healing of Concave Wounds

In one embodiment, a method for promoting the healing of a concave wound may comprise placing a scaffold as described herein over the concave wound. The method may also include pressing the scaffold into the concave wound, such that the scaffold simultaneously or substantially simultaneously contacts at least a bottom portion of the concave wound and at least a side portion of the concave wound. In some embodiments, the scaffold may simultaneously or substantially simultaneously contact all or substantially all of the exposed portions of the concave wound.

In certain embodiments, the scaffold may be delivered to or near the concave wound using a delivery device. The delivery device may be, for example, a plunger or cylinder configured to place the scaffold into the concave wound. In one embodiment, the delivery device may also be configured to press the scaffold into the concave wound, as described herein.

In some embodiments, the step of pressing the scaffold into the concave wound may comprise pressing the center (or approximately the center) of the scaffold into the bottom portion of the concave wound, and allowing a portion of the scaffold to fold upward to contact the side portion of the concave wound. In embodiments wherein the scaffold comprises a shape of a cupcake liner, as described herein the step of pressing the scaffold into the concave wound may comprise pressing the center of the scaffold into the bottom portion of the concave wound, and allowing the at least one wall of the scaffold to contact the side portion of the concave wound. In embodiments wherein the scaffold comprises a shape of an asterisk, as described herein, the step of pressing the scaffold into the concave wound may comprise pressing the center of the scaffold into the bottom portion of the concave wound, and allowing at least two of the spokes of the scaffold to fold upward to contact the side portion of the concave wound.

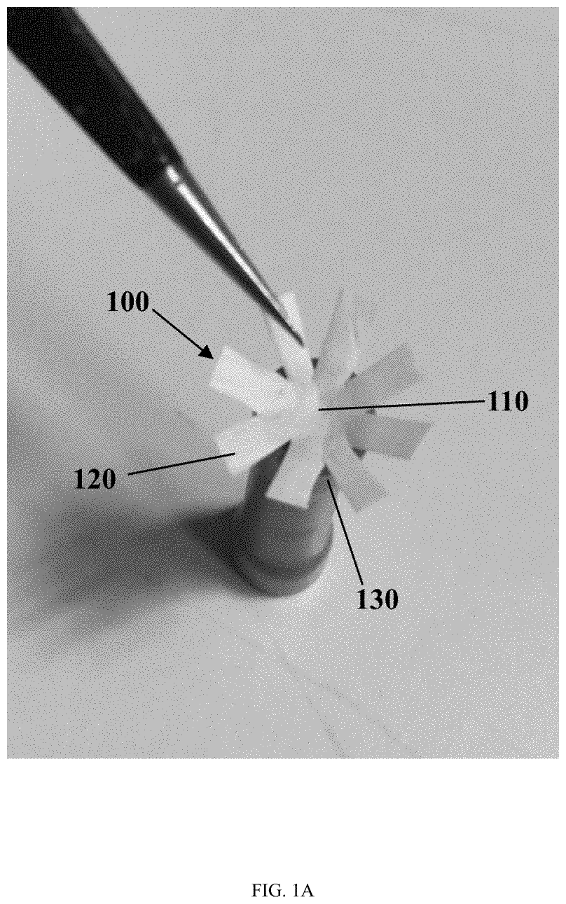

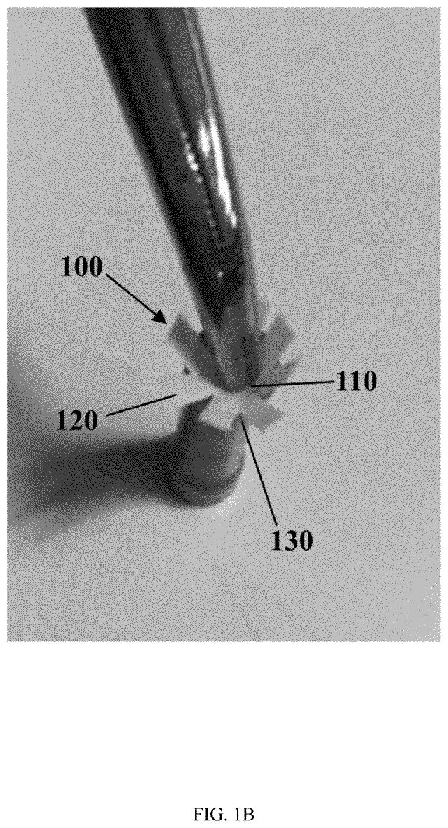

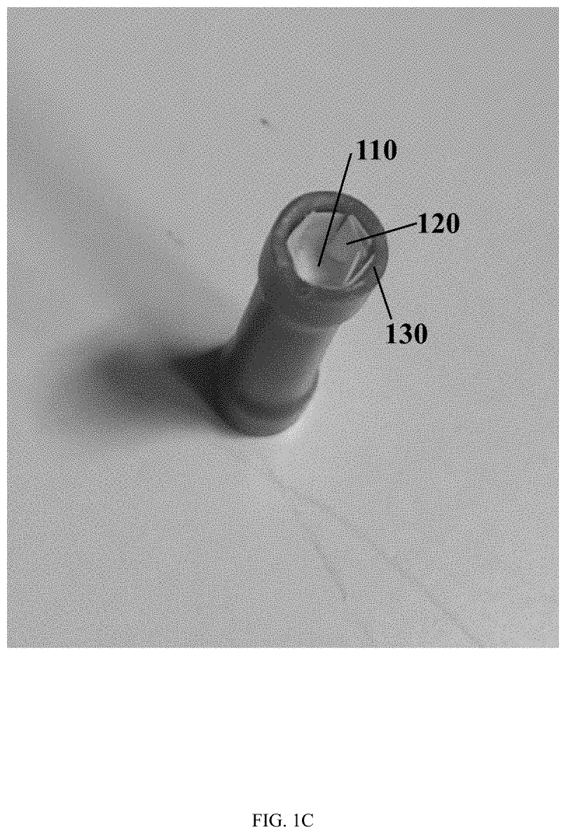

FIG. 1A, FIG. 1B, and FIG. 1C illustrate various embodiments of a method of promoting the healing of a concave wound, as described herein. FIG. 1A illustrates an embodiment of the step of placing a scaffold 100 over a model of a concave wound 130, as described herein. The model of the concave wound 130 is a model of a punch biopsy site, as described herein. The scaffold 100 has a shape of an asterisk, the asterisk comprising a center 110 and eight spokes 120, as described herein. FIG. 1B illustrates an embodiment of the step of pressing the scaffold 100 having a center 110 and spokes 120 into the model of the concave wound 130, as described herein. FIG. 1C illustrates an embodiment of the scaffold 100 simultaneously contacting at least a bottom portion of the model of the concave wound 130 and at least a side portion of the model of the concave wound 130, as described herein. Specifically, the center 110 of the scaffold 100 is contacting the bottom of the concave wound 130, and simultaneously the spokes 120 of the scaffold 100 are contacting at least one side of the concave wound 130, in accordance with the present disclosure.

Without wishing to be bound by theory, employing scaffolds as described herein by methods described herein may provide a high surface area interface with one or more surfaces of the concave wound, and the interface may encourage cell and tissue adhesion and remodeling, ultimately promoting the healing of the concave wound. The unique shapes of the scaffolds described herein may allow the scaffold to uniformly or substantially uniformly contact the bed of the concave wounds described herein, whereas a flat sheet or other dressing may not be able to achieve that level of contact. The scaffolds as described herein are soft, flexible, and easily draped over a wound bed, while also being easy to shape into specific shapes to maximize wound coverage. The electrospun polymer fibers of such a scaffold may allow for a high degree of flexibility, allowing the scaffold to be easily manipulated by a physician or other healthcare provider during placement and usage. Furthermore, the ease with which a scaffold may be manipulated may make it more comfortable for patients or subjects, increasing their compliance with its use. In addition, using the scaffolds described herein may eliminate or reduce the need for wound dressing changes, which are often painful. Employing scaffolds as described herein may also, for example, help mimic native extracellular matrix morphology, help promote wound healing through all healing phases (without stalling in the inflammatory phase), wick moisture, facilitate cell adherence and infiltration, support capillary growth and cellular proliferation, support native mechanochemical behavior (including intercellular communication), and help regenerate fully functional tissue.

In certain embodiments, the methods described herein may be suture-free. In other words, a physician or other medical professional may apply a scaffold using the methods described herein, and may not need to secure the scaffold in place with a suture or any other type of securing procedure. In one embodiment, a suture-free method for promoting the healing of a concave wound may provide an advantage over traditional healing or treatment techniques, because it may allow improved healing combined with fewer return visits to a patient's physician or other healthcare provider. When a method for promoting the healing of concave wounds is suture-free, a patient need not return to a physician's office for the simple removal of a suture, for example.

In some embodiments, the methods described herein may result in the scaffold substantially uniformly contacting the bed of the concave wound. In other embodiments, the concave wounds may heal more rapidly than with traditional management methods because, without wishing to be bound by theory, the scaffolds described herein may promote improved healing with reduced scarring. This may be, for example, a result of the use of fewer foreign materials such as sutures in the wounds. Without wishing to be bound by theory, this may also be a result of the scaffolds herein promoting or assisting with hemostasis, application, inflammation, proliferation, and/or remodeling of the concave wound, as described herein. While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1.-32. (canceled)

33. A method for promoting the healing of a concave wound, the method comprising:
placing a scaffold over the concave wound, the scaffold comprising an electrospun polymer fiber; and
pressing the scaffold into the concave wound, such that the scaffold simultaneously contacts at least a bottom portion of the concave wound and at least a side portion of the concave wound.

34. The method of claim 33, wherein the method is suture-free.

35. The method of claim 33, wherein the concave wound is selected from the group consisting of a punch biopsy site, a puncture wound, a cavernous wound, a traumatic wound, and combinations thereof.

36. The method of claim 33, wherein the scaffold has an asterisk shape comprising a center and at least two spokes.

37. The method of claim 36, wherein pressing the scaffold into the concave wound comprises pressing the center of the scaffold into the bottom portion of the concave wound, and allowing the at least two spokes of the scaffold to fold upward to contact the side portion of the concave wound.

38. The method of claim 36, wherein the center of the scaffold having the asterisk shape has a diameter of about 1 mm to about 300 mm, and wherein the at least two spokes of the scaffold having the asterisk shape each have a length of about 1 mm to about 300 mm.

39. The method of claim 33, wherein the scaffold has a cupcake liner shape comprising a flat center and at least one wall substantially perpendicular to the center.

40. The method of claim 39, wherein pressing the scaffold into the concave wound comprises pressing the center of the scaffold into the bottom portion of the concave wound, and allowing the at least one wall of the scaffold to contact the side portion of the concave wound.

41. The method of claim 39, wherein the center of the scaffold having the cupcake liner shape has a diameter of about 1 mm to about 300 mm, and wherein the at least one wall of the scaffold having the cupcake liner shape has a height of about 1 mm to about 300 mm.

42. The method of claim 39, wherein the center of the scaffold having the cupcake liner shape has a diameter of about 4 mm, and wherein the at least one wall of the scaffold having the cupcake liner shape has a height of about 4 mm.

43. The method of claim 33, wherein the electrospun polymer fiber comprises at least two electrospun polymer fibers, each electrospun polymer fiber comprising a polymer independently selected from the group consisting of polyethylene oxide terephthalate, polybutylene terephthalate, polyethylene oxide terephthalate-co-polybutylene terephthalate, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof, and wherein the at least two electrospun polymer fibers are co-electrospun.

44. The method of claim 33, wherein the depth of the concave wound is at least about 1 mm.

45. A scaffold for promoting the healing of an concave wound, the scaffold comprising an electrospun polymer fiber and having a shape selected from the group consisting of an asterisk, a cupcake liner, and combinations thereof.

46. The scaffold of claim 45, wherein the scaffold is configured to simultaneously contact at least a bottom portion of the concave wound and at least a side portion of the concave wound.

47. The scaffold of claim 45, wherein the concave wound is selected from the group consisting of a punch biopsy site, a puncture wound, a cavernous wound, a traumatic wound, and combinations thereof.

48. The scaffold of claim 45, wherein the scaffold has a shape of an asterisk comprising a center and at least two spokes.

49. The scaffold of claim 48, wherein the center has a diameter of about 1 mm to about 300 mm, and wherein the at least two spokes each have a length of about 1 mm to about 300 mm.

50. The scaffold of claim 45, wherein the scaffold has a shape of a cupcake liner comprising a flat center and at least one wall substantially perpendicular to the center.

51. The scaffold of claim 50, wherein the center of the scaffold has a diameter of about 1 mm to about 300 mm, and wherein the at least one wall of the scaffold has a height of about 1 mm to about 300 mm.

52. The scaffold of claim 50, wherein the center of the scaffold has a diameter of about 4 mm, and wherein the at least one wall of the scaffold has a height of about 4 mm.
